(19) United States
(12) Patent Application Publication
(10) Pub. No.: US 2007/0197431 A1
(43) Pub. Date: Aug. 23, 2007

(54) Novel protein for inhibiting tumor progression and increasing nerve regeneration

(76) Inventors: Kermit Carraway, Sacramento, CA (US); Alfred-John Diamonti, Carmichael, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 10/370,191

(22) Filed: Feb. 20, 2003

Related U.S. Application Data
(60) Provisional application No. 60/358,793, filed on Feb. 21, 2002.
(60) Provisional application No. 60/378,570, filed on May 7, 2002.

(51) Int. Cl. A61K 38/17; A61K 48/00; C07K 14/575; C07H 21/04; C12P 21/06

(52) U.S. Cl. 514/12; 514/44; 435/69.1; 435/320.1; 435/325; 530/350; 536/23.5

(57) ABSTRACT

The present invention provides compositions and methods useful for inhibiting the activity of ErbB3 and ErbB4, inhibiting neoplastic growth, or increasing nerve growth. The present invention also provides a dominant negative form of Nrdp1 and methods of use thereof. Methods screening for agents capable of regulating the interaction of Nrdp1 with ErbB3, ErbB4, or nerve regeneration inhibitors are also provided by the present invention.

### CROSS REFERENCE TO RELATED APPLICATIONS

The present application claims priority under 35 U.S.C. §119(e) from provisional application Nos. 60/358,793, filed Feb. 21, 2002; and 60/378,570, filed May 7, 2002.

### ACKNOWLEDGEMENT OF GOVERNMENT INTEREST

This invention was made in part with government support under Grant No. NIH/NCI CA71701 awarded by the National Institutes of Health (NIH). The government may have certain rights in this invention.

### FIELD OF THE INVENTION

This invention relates generally to the field of inhibition of tumor growth and enhancement of nerve regeneration or growth.

### BACKGROUND OF THE INVENTION

The neuregulins comprise a subfamily of at least four epidermal growth factor (EGF)-like growth factors that influence a variety of cellular events, including proliferation, differentiation, migration, survival and fate. The most thoroughly examined neuregulin, neuregulin-1 (NRG1; also called heregulin, neu differentiation factor, glial growth factor or acetylcholine receptor inducing activity), has been shown to play essential roles in the development of cardiac and neural tissues, and has also been shown to play a role in the postsynaptic development of the neuromuscular junction (Burden, S., et al., Neuron, 18:847-55 (1997)). The neuregulins bind to ErbB3 or ErbB4, members of the EGF receptor family of receptor tyrosine kinases, and can activate the ErbB2 receptor through a heterodimerization mechanism (Carraway, K. L., III, et al., Cell, 78:5-8; Alroy, I., et al., FEBS Lett., 410:83-86 (1997); Riese, D. J., II, et al., Bioessays, 20:41-48; and Olayioye, M. A., et al., EMBO J, 19:3159-67 (2000)).

Recent studies suggest that the fidelity of signaling through ErbB and other growth factor receptors is ensured by the maintenance of a relatively narrow range of receptors at the cell surface site of signaling (Carraway, K. L., III, et al., Curr. Opin. Cell Biol., 13:125-30 (2001)). For example, in the nematode Caenerhabditis elegans mutations in the proteins lin-2 or lin-7 mislocalize the worm ErbB receptor let-23 and abrogate its function in vulval cell fate determination (Simske, J. S., et al., Cell, 85:195-04 (1996)). Overexpression of the let-23 receptor can compensate for the mislocalization defect, suggesting that the accumulation of threshold levels of ErbB receptors at a specific cell surface location is necessary for biological activity. However, receptor overexpression can also lead to disease states such as the genesis or progression of tumors. Hence, mechanisms must exist for localizing and maintaining a precise concentration of growth factor receptors at the site of signal reception.

Interestingly, it has been reported that ErbB2, ErbB3 and ErbB4 are somewhat unique among examined receptor tyrosine kinases in that they exhibit impaired ligand-induced internalization, down-regulation and degradation (Baulida, J., et al., J. Biol. Chem., 271:5251-57(1996) and Baulida, J., et al., Exp. Cell Res., 232:167-72)), underscoring the need for very tight regulation of cell surface receptor concentration prior to ligand stimulation. Conceptually, steady-state levels of cell surface proteins are maintained as a result of a balance between synthesis, delivery, retention and degradation. While synthesis, delivery and retention play crucial roles in establishing the pattern of cell surface protein expression, degradation is necessary to remove overproduced or mislocalized proteins and to maintain the dynamic equilibrium of expressed protein. Anchoring or trafficking proteins involved in these processes are predicted to interact with receptors in an activation-independent manner.

Past early childhood, injury to the central nervous system (CNS) results in functional impairments that are largely irreversible. Within the brain or spinal cord, damage resulting from stroke, trauma, or other causes can result in life-long losses in cognitive, sensory and motor functions, and even maintenance of vital functions. Nerve cells that are lost are not replaced, and those that are spared are generally unable to regrow severed connections, although a limited amount of local synaptic reorganization can occur close to the site of injury. Functions that are lost are currently untreatable. Regenerative failure in the CNS has been attributed to a number of factors, which include the presence of inhibitory molecules on the surface of glial cells that suppress axonal growth; absence of appropriate substrate molecules such as laminin to foster growth; and an absence of the appropriate trophic factors needed to activate programs of gene expression required for cell survival and differentiation.

There is a need in the art to provide methods and compositions useful for inhibiting tumor growth and increasing nerve regeneration or growth.

### SUMMARY OF THE INVENTION

The present invention is based, in part, on the discovery that Nrdp1 can decrease the activity of ErbB3 and ErbB4 and interact with inhibitors of nerve regeneration, e.g., Nogo-A. The present invention provides compositions and methods useful for inhibiting the activity of ErbB3 and ErbB4, inhibiting neoplastic growth, or increasing nerve growth. The present invention also provides a dominant negative form of Nrdp1 and methods of use thereof. Methods screening for agents capable of regulating the interaction of Nrdp1 with ErbB3, ErbB4, or nerve regeneration inhibitors are also provided by the present invention.

In one embodiment, the present invention provides an isolated polypeptide which comprises an Nrdp1 amino acid sequence consisting of an ErbB3 binding domain of Nrdp1, wherein the Nrdp1 amino acid sequence is not adjacent to an amino acid sequence to which it is naturally adjacent.

In another embodiment, the present invention provides a composition which comprises Nrdp1 or a polynucleotide encoding Nrdp1 and a pharmaceutically acceptable carrier.

In yet another embodiment, the present invention provides a method for decreasing the activity of ErbB3 or ErbB4 in a cell. The method comprises contacting the cell with Nrdp1 or introducing a polynucleotide sequence encoding Nrdp1 to the cell.

In still another embodiment, the present invention provides a method for treating the neoplastic growth of a cell. The method comprises contacting the cell with Nrdp1 or introducing a polynucleotide sequence encoding Nrdp1 to the cell.

In another embodiment, the present invention provides a method for treating a neoplastic growth. The method comprises administering to a subject in need of such treatment an effective amount of Nrdp1 or a polynucleotide sequence encoding Nrdp1.

In another embodiment, the present invention provides a method for increasing neuregulin stimulation in a cell. The method comprises contacting the cell with an isolated polypeptide containing the ErbB3 binding domain of Nrdp1 as provided by the present invention.

In another embodiment, the present invention provides a method of screening for an agent capable of regulating an interaction between ErbB3 and Nrdp1. The method comprises contacting ErbB3 and Nrdp1 in the presence of a test agent, wherein a change in the interaction between ErbB3 and Nrdp1 caused by a test agent is indicative that the agent is capable of regulating the interaction between ErbB3 and Nrdp1.

In another embodiment, the present invention provides a method of screening for an agent capable of regulating an interaction between Nogo-A and Nrdp1. The method comprises contacting Nogo-A and Nrdp1 in the presence of a test agent, wherein a change in the interaction between Nogo-A and Nrdp1 caused by a test agent is indicative that the agent is capable of regulating the interaction between Nogo-A and Nrdp1.

In yet another embodiment, the present invention provides a method for increasing nerve growth. The method comprises administering to a subject in need of such treatment an effective amount of Nrdp1 or a polynucleotide sequence encoding Nrdp1.

In still another embodiment, the present invention provides a method for inhibiting the activity of Nogo-A in a cell. The method comprises contacting the cell with Nrdp1 or a polynucleotide sequence encoding Nrdp1 to the cell.

### SUMMARY OF THE FIGURES

FIG. 1 shows the molecular characterization of Nrdp1. The amino acid sequence of the full-length RBCC protein Nrdp1 (SEQ ID NO. 3) is depicted. The imperfect C3HC4 RING finger domain is shaded, the Bbox region is underlined and the coiled-coil region is italicized.

FIG. 2 shows the FLAG-tagged constructs employed in the studies.

### DESCRIPTION OF THE PREFERRED EMBODIMENTS

The present invention is based, in part, on the discovery of Nrdp1 and its ability to decrease the activity of ErbB3 and ErbB4, inhibit neoplastic growth, and interact with nerve regeneration inhibitors. The present invention provides compositions and methods for inhibiting the activity of ErbB3 and ErbB4, inhibiting neoplastic growth, and enhancing nerve regeneration or growth. The present invention also provides dominant-negative forms of Nrdp1 and the use thereof. Methods for screening for agents capable of regulating the interaction of Nrdp1 with ErbB3, ErbB4, and nerve regeneration inhibitors are also provided by the present invention.

One aspect of the present invention provides compositions of Nrdp1. According to the present invention, Nrdp1 is a member of RBCC subfamily of RING finger-containing proteins and is involved in neuregulin receptor degradation. Nrdp1 usually contains a RING finger domain, B-box domain, coiled-coil domain, and ErbB3 binding domain.

In one embodiment, Nrdp1 includes a polypeptide having the amino acid sequence as shown in SEQ ID NO. 3. In another embodiment, Nrdp1 includes mutated, truncated, or modified Nrdp1, or derivatives thereof that having one or more functions of the polypeptide with the amino acid sequence as shown in SEQ ID NO. 3. In yet another embodiment, Nrdp1 includes polypeptides having an amino acid sequence with at least 99%, 95%, 90%, 85%, 80%, 70%, or 60% identity of the sequence as shown in SEQ ID NO. 3. In still another embodiment, Nrdp1 includes mutated, truncated, or modified Nrdp1, wherein the mutation, truncation, or modification occurs outside of one or more functional domains, e.g., RING finger domain, B-box domain, coiled-coil domain, or ErbB3 binding domain of Nrdp1. According to the present invention, Nrdp1 of the present invention can be from any species, e.g., human or its orthologs from other species such as mouse.

The functional domains of Nrdp1 can be defined by sequence, function, or both. For example, the consensus sequence for RING finger domain, B-box domain, and coiled-coil domain are known to one skilled in the art and can be used to define these domains within Nrdp1. In one embodiment, the RING finger domain includes amino acids from position 18 to 56 of SEQ ID NO. 3, the B-box domain includes amino acids from position 83 to 134 of SEQ ID NO. 3, and the coiled-coil domain includes amino acids from position 138 to 181 of SEQ ID NO.3.

Another aspect of the present invention provides isolated peptides containing the ErbB3 binding domain of Nrdp1 and compositions thereof. The ErbB3 binding domain of Nrdp1 includes any truncated Nrdp1 that is capable of binding to ErbB3 or ErbB4. Usually such binding is constitutive and independent of the activation of ErbB3 or ErbB4. In one embodiment, the ErbB3 binding domain includes a truncated Nrdp1 lacking any one of the RING finger domain, B-box domain, and coiled-coil domain. In another embodiment, the ErbB3 binding domain includes a truncated Nrdp1 lacking any two of the RING finger domain, B-box domain, and coiled-coil domain. In yet another embodiment, the ErbB3 domain includes a truncated Nrdp1 lacking the RING finger domain, B-box domain, and coiled-coil domain.

According to the present invention, the ErbB3 binding domain of Nrdp1 can also include any polypeptide containing at least 138 or 183 C-terminal amino acids of Nrdp1 or containing the amino acid sequence as shown in SEQ ID NO. 1 or SEQ ID NO. 2. In one embodiment, the ErbB3 binding domain is not surrounded by or adjacent to any amino acid sequences that are naturally adjacent to the ErbB3 binding domain.

Another feature of the present invention provides isolated crystalline polypeptides containing Nrdp1 or the ErbB3 binding domain of Nrdp1. The crystalline polypeptides can be made by any suitable means known to one skilled in the art. For example, polypeptides containing Nrdp1 or the ErbB3 binding domain of Nrdp1 can be crystallized by equilibrating saturated solutions of the polypeptides with salts, volatile organic compounds, and other organic compounds at various controlled temperatures.

Yet another feature of the present invention provides antibodies, e.g., isolated monoclonal antibodies that specifically bind to an epitope within Nrdp1 or the ErbB3 binding domain of Nrdp1. Such antibodies can be prepared by any means known to one skilled in the art. For example, whole or partial amino acid sequences of Nrdp1 or the ErbB3 binding domain of Nrdp1 can be used as an antigen to obtain monoclonal antibodies.

Yet another feature of the present invention provides isolated polynucleotides encoding Nrdp1 or the ErbB3 binding domain of Nrdp1. These polynucleotides can be included in a vector suitable for expression of Nrdp1 or the ErbB3 binding domain of Nrdp1 in an appropriate expression system, e.g., a bacterial, yeast, plant, or eukaryotic cell expression system. Polynucleotides encoding Nrdp1 or the ErbB3 binding domain of Nrdp1 can also be included in a vector, e.g., adenoviral or retroviral vector suitable for gene delivery in vitro or in vivo. In addition, cells can be transformed or transfected by the polynucleotides encoding Nrdp1 or the ErbB3 binding domain of Nrdp1.

The compositions of Nrdp1 or the ErbB3 binding domain of Nrdp1 provided by the present invention usually include a pharmaceutically acceptable carrier and Nrdp1 or the ErbB3 binding domain of Nrdp1. Pharmaceutically acceptable carriers are well known to those in the art. Such carriers include, without limitation, large, slowly metabolized macromolecules, e.g., proteins, polysaccharides, polylactic acids, polyglycolic acids, polymeric amino acids, amino acid copolymers, and inactive virus particles.

Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition can also contain liquids, e.g., water, saline, glycerol, and ethanol, as well as substances, e.g., wetting agents, emulsifying agents, or pH buffering agents. In addition, liposomes or other delivery particles can also be used as a carrier for the compositions of the present invention.

The polypeptides, polynucleotides, vectors, cells, and compositions thereof provided by the present invention are useful for 1) regulating the activity of ErbB3 or ErbB4 and the signal transduction via ErbB3 or ErbB4, 2) treating the neoplastic growth of a cell, and 3) regulating neuregulin stimulation in a cell. For example, Nrdp1 of the present invention and its encoding polynucleotides are useful for decreasing the activity of ErbB3 or ErbB4 in a cell. In one embodiment, Nrdp1 decreases the activity of ErbB3 or ErbB4 by decreasing or suppressing the expression of ErbB3 or ErbB4, e.g., decreasing the steady state level of ErbB3 or ErbB4. In another embodiment, Nrdp1 decreases the amount of ErbB3 on the surface of a cell, e.g., by redistributing ErbB3 to intracellular Nrdp1-containing compartments. In yet another embodiment, Nrdp1 decreases the activity of ErbB3 or ErbB4 without causing a detectable change of the activity of EGF receptor or ErbB2.

Nrdp1 of the present invention and its encoding polynucleotides are also useful for treating neoplasia. For example, Nrdp1 or its encoding polynucleotide can be contacted with or introduced to a cell or administered to a subject in need of such treatment to inhibit or decrease a neoplastic growth. In one embodiment, Nrdp1 or its encoding polynucleotide is used to treat a neoplastic growth that is HER2/neu-positive. In another embodiment, Nrdp1 or its encoding polynucleotide is used to treat solid tumors, e.g., HER2/neu-positive tumors including without limitation, breast, ovary, prostate, and lung tumors.

According to the present invention, polypeptides containing the ErbB3 binding domain of Nrdp1 or its encoding polynucleotide can also be used to increase neuregulin stimulation in a cell by contacting the cell with or introducing to the cell polypeptides containing the ErbB3 binding domain of Nrdp1 or its encoding polynucleotide.

According to another aspect of the present invention, Nrdp1 of the present invention is useful for inhibiting nerve regeneration inhibitors. For example, Nrdp1 can be used to inhibit the activity of nerve regeneration inhibitors, e.g., via binding or interacting with a nerve regeneration inhibitor. Any nerve regeneration inhibitor, known or later discovered, can be suitable target for Nrdp1, e.g., Nogo-A, a major inhibitor of nerve regeneration in adults.

In one embodiment, Nrdp1 of the present invention or its encoding polynucleotide is used to inhibit the activity of nerve regeneration inhibitors or increase nerve growth. For example, Nrdp1 or its encoding polynucleotide can be introduced to cells or administered to a subject in need of such treatment to inhibit or decrease the activity of nerve regeneration inhibitors, the activity of Nogo-A, or increase nerve growth.

The polypeptides and the compositions of the present invention useful for therapeutic treatment can be administered alone, in a composition with a suitable pharmaceutical carrier, or in combination with other therapeutic agents. An effective amount of the polypeptides or the compositions of the present invention to be administered can be determined on a case-by-case basis. Factors should be considered usually include age, body weight, stage of the condition, other disease conditions, duration of the treatment, and the response to the initial treatment.

Typically, the polypeptides or the compositions of the present invention are prepared as an injectable, either as a liquid solution or suspension. However, solid forms suitable for solution in, or suspension in, liquid vehicles prior to injection can also be prepared. The polypeptides or the compositions of the present invention can also be formulated into an enteric-coated tablet or gel capsule according to known methods in the art.

The polypeptides of the compositions of the present invention may be administered in any way which is medically acceptable which may depend on the disease condition or injury being treated. Possible administration routes include injections, by parenteral routes such as intravascular, intravenous, intraepidural or others, as well as oral, nasal, ophthalmic, rectal, topical, or pulmonary, e.g., by inhalation. The polypeptides or the compositions of the present invention may also be directly applied to tissue surfaces, e.g., during surgery. Sustained release administration is also specifically included in the invention, by such means as depot injections or erodible implants.

The present invention also provides methods for screening for an agent capable of regulating the interactions between Nrdp1 and ErbB3 or Nogo-A. In one embodiment, the method includes contacting ErbB3 or Nogo-A with Nrdp1 in the presence of a test agent and measuring the interaction between ErbB3 or Nogo-A with Nrdp1, e.g., measuring the binding activity between ErbB3 or Nogo-A with Nrdp1. A test agent that changes the interaction between ErbB3 or Nogo-A with Nrdp1 is indicative of an agent capable of regulating the interaction between ErbB3 or Nogo-A with Nrdp-1.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

In a screen for proteins that might regulate the trafficking or localization of the ErbB3 receptor, we have identified a novel tripartite or RBCC (RING, B-box, coiled-coil) protein that interacts with the cytoplasmic tail of the receptor in an activation-independent manner. We have named this protein Nrdp1 for neuregulin receptor degradation protein-1. Northern blotting reveals ubiquitous distribution of Nrdp1 in human adult tissues, but message is particularly prominent in heart, brain and skeletal muscle. Nrdp1 interacts specifically with the neuregulin receptors ErbB3 and ErbB4 and not with EGF receptor or ErbB2. When co-expressed in COS7 cells, Nrdp1 mediates the redistribution of ErbB3 from the cell surface to intracellular compartments, and induces the suppression of ErbB3 and ErbB4 receptor levels but not EGF receptor or ErbB2 levels. A dominant-negative form of Nrdp1 potentiates neuregulin-stimulated Erk1/2 activity in transfected MCF7 breast tumor cells. We believe that Nrdp1 acts to regulate steady-state cell surface neuregulin receptor levels, thereby influencing the efficiency of neuregulin signaling.

### Example 1

### Yeast Two-Hybrid Screens, Cloning, Constructs, and Antibody Preparation

The region of cDNA encoding the entire intracellular portion of bovine ErbB3 (residues W665 to I1335) was amplified and subcloned into the pAS1 bait plasmid. This construct was co-transformed with a plasmid library encoding human brain cDNAs fused to the activation domain of GAL4 into the Y190 lacZ/HIS3 yeast reporter strain. His⁺/β-gal⁺ clones were recovered and sequenced. Carboxy terminal deletions of the ErbB3 intracellular domain in the same plasmid were cotransformed into yeast with the pGAD plasmid containing no insert or the clone 32 insert (see below), and colonies were scored for β-galactosidase activity.

The ˜650 bp clone 32 from the two hybrid screen was subcloned into the EcoRI site of pGEX to create a GST fusion protein encompassing Nrdp1 residues I135 to I317. Northern blotting was carried out using clone 32 to probe a filter of adult human tissues (Clontech). This clone was also used to screen a λgtll human fetal brain library to obtain ˜3.7 kb and ˜2.2 kb clones, which were partially sequenced. The cDNA sequence and open reading frame were found to be identical to the unpublished hypothetical protein SBBI03 (GenBank accession number AF077599), and highly homologous to the mouse FLRF protein (GenBank accession number AF305730). Four PCR primers were employed to generate the full-length protein (M1 to I317), the amino terminal Zn²⁺-binding domains (M1 to D169)and the clone 32 region, each tagged with the FLAG epitope (DYKDDDDK) (SEQ ID NO. 4) at the carboxy terminus. These were subcloned into the NotI and XbaI sites of mammalian expression plasmid pcDNA3.1 (Invitrogen). A similar PCR approach was used to FLAG tag Nrdp1 at the amino terminus, and this construct was subcloned into baculovirus transfer vector pVL1392 for insect cell expression. cDNAs encoding human ErbB2 and ErbB4 were independently subcloned into expression vector pcDNA3. 1, and the vector encoding bovine ErbB3 was described previously (Carraway, K. L., III, et al., J. Biol. Chem., 270:7111-16 (1995)).

A polyclonal rabbit antibody was raised to the bacterially-expressed GST fusion protein of clone 32 described above by Research Genetics, Inc. Anti-Nrdp1 antibodies were affinity purified from serum obtained from week 10 bleeds using the fusion protein immobilized on Affi-Gel 10 beads (Bio-Rad) according to procedures outlined previously (Carraway, K. L., III, et al., J. Biol. Chem., 270:7111-16 (1995)).

### Example 2

### Mammalian Cells, Transfections and Protein Expression

COS7 or MCF7 cells from ATCC were maintained in DMEM/10% fetal calf serum at 10% CO2. For transfection experiments cells were grown to 50% confluence in 6-well dishes and transfected with 2 μg total DNA and 10 μl Superfect Qiagen according to the protocol outlined by the manufacturer. For stable transfections, cells were split into 100 mm dishes and selected in 0.4 mg/ml G418 (Gibco). In transient transfection experiments, pcDNA3.1 plasmid was used in mock transfections and in cotransfections not requiring a second component. Following transfection, cells were allowed to recover for 30 hours, and then were treated without or with 2 μM MG132 (Novabiochem) for 18 hours. Cells were lysed in an NP-40-containing lysis buffer (Crovello, C. S., et al., J. Biol. Chem., 273:26954-61 (1998)) or RIPA, or in 1× sample buffer.

For immunoprecipitation experiments, cleared lysates were precipitated with anti-EGF receptor (Ab1), anti-ErbB2 (Ab4), anti-ErbB3 (Ab6)or anti-ErbB4 (Ab1), all from NeoMarkers. Blotting antibodies used were: anti-EGFR (1005, Santa Cruz), anti-ErbB2 (Ab1, NeoMarkers), antiErbB3 (3184; 10), anti-ErbB4 (Ab2, NeoMarkers), and anti-FLAG antibody M2 (Sigma).

Chemiluminescent signal from HRP-conjugated secondary antibodies was detected using an AlphaInnotech 8000 imaging station with FluorChem software. For Erk stimulation experiments, transfected MCF7 cells were treated without or with various concentrations of NRG1β (Carraway, K. L., III, et al., Nature, 387:512-16 (1997)) 15 minutes prior to lysis with 1× sample buffer. Lysates were resolved using 6-10% linear gradient gels and transferred to nitrocellulose. Blots were probed with anti-ErbB3 Ab6, anti-FLAG, anti-HA tag (Boehringer-Mannheim) or anti-phospho-Erk1/2 (Cell Signaling Technology).

### Example 3

### Insect Cell and GST Fusion Protein Pull-Down Experiments

Baculovirus encoding Nrdp1 tagged at its amino terminus with FLAG epitope was created as described previously (Guy, P. M., et al., Proc. Natl. Acad. Sci USA., 91:8132-36 ((1994)). Baculoviruses encoding human EGF receptor, human ErbB2, bovine ErbB3 or human ErbB4 have been described previously (Carraway, K. L., III, et al., Nature, 387:512-16 (1997)). For co-immunoprecipitation experiments Sf9 cells were infected with receptor viruses and co-infected either wild type or Nrdp1 baculovirus. 48 hours after infection cleared NP-40 lysates were immunoprecipitated with receptor antibodies and blotted with antibodies directed to receptors or FLAG epitope as described above.

For pull-down experiments receptors were expressed in Sf9 insect cells, cleared lysates were incubated for 1.5 hours with 3 μg immobilized GST or GST-32, and beads were washed as previously described (Carraway, K. L., III, et al., Nature, 387:512-16 (1997)). MDA-MB-453 cells were treated for 5 minutes without or with 10 nM NRG1β, lysed as previously described (Sweeney, C., et al., J. Biol. Chem., 275:19803-07 (2000)), and cleared lysates were incubated with GST and GST-32 as above. Precipitated proteins were resolved by 8% SDS-PAGE, transferred to nitrocellulose and visualized by blotting with anti-phosphotyrosine (RC20, Transduction Laboratories) or anti-receptor antibodies.

### Example 4

### Immunofluorescence

COS cells grown to 50% confluence on cover slips were transfected as described above, and fixed in methanol after 48 hours. Cover slips were blocked in 1% BSA, 0.2% NP-40, 5% goat serum, 0.02% sodium azide, and then incubated for 1 hour in primary antibody followed by 1 hour in secondary antibody. Primary antibodies used were 1/500 antiFLAG M2, 1/500 anti-ErbB3 Ab6 (NeoMarkers)and 1 μg/ml affinity-purified anti-Nrdp1. The secondary antibodies used were 1/200 FITC-goat anti-mouse IgG and 1/200 Cy3-goat anti-rabbit IgG, both from Jackson Immunologicals. Cover slips were mounted onto glass slides using Fluoromount-G (Southern Biotechnology Associates). Confocal microscopy at 60× magnification was carried out using a BioRad MRC 600 confocal microscope with an argon laser. For each transfectant, 24 images were collected corresponding to focal planes encompassing the entire depth of the cell.

### Example 5

### Isolation and Characterization of Nrdp1

The localization of ErbB receptors and the regulation of their levels play critical roles in the fidelity of growth factor signal transduction (Carraway, K., et al., Curr. Opin. Cell Biol., 13:125-30)). Proteins involved in these processes are predicted to interact with receptors in an activation-independent manner. Since ErbB3 lacks intrinsic tyrosine kinase and autophosphorylation activities (Guy, P. M., et al., Proc. Natl. Acad. Sci. USA, 91:8132-36 (1994)), proteins that interact with the intracellular portion of this receptor in a yeast two-hybrid screen bind in a phosphotyrosine- and activation-independent manner. Using the entire intracellular region of ErbB3 as bait, we identified a ˜650 bp cDNA fragment (called clone 32) in a human fetal brain library that encodes an interacting polypeptide. We further identified a 120 amino acid sequence in the ErbB3 tail region (T1159-E1279) necessary for clone 32 association with bovine ErbB3.

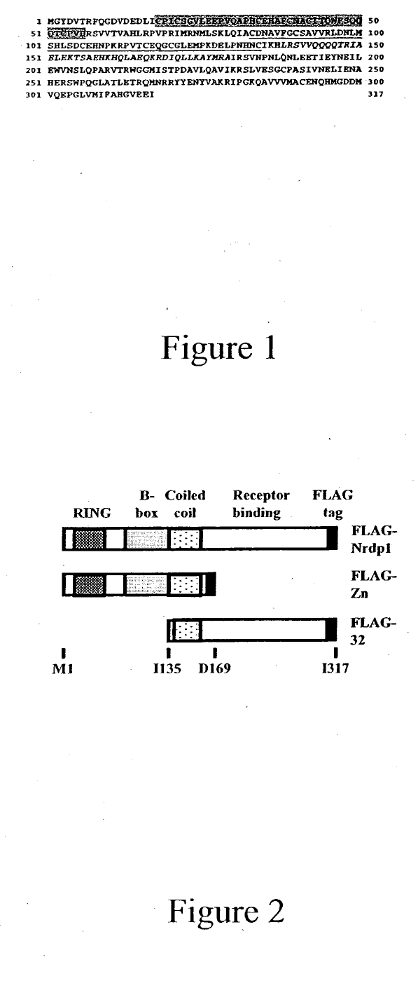

Sequencing of clone 32 revealed that it encodes the carboxy-terminal 183 amino acids of human hypothetical protein SBBI03 (GenBank accession number AF077599) and its mouse ortholog FLRF (accession number AF305730), proteins of unknown biochemical or cellular function (Abdullah, J. M., et al., Blood Cells Mol. Dis., 27:320-33 (2001)). The complete amino acid sequence of this protein (FIG. 1) indicates that it is a member of the RBCC subfamily of RING finger-containing proteins. The presence of a putative myristoylation site at its amino terminus implies a membrane function for Nrdp1. On the basis of its homology with RING finger proteins known to be involved in the degradation of cell surface proteins, and on our functional characterization below, we have named the protein Nrdp1 for neuregulin receptor degradation protein-1. Although the message appears to be ubiquitously expressed in adult human tissues, prominent Nrdp1 expression in heart, brain and skeletal muscle is consistent with the known roles for neuregulin signaling in the development and maintenance of these tissues.

To begin to characterize the function of the Nrdp1 protein we expressed epitope (FLAG)-tagged forms of the full-length protein and its two halves FLAG-RING and FLAG-32 (see FIG. 2) in a variety of mammalian cell lines. The FLAG tag was placed at the carboxy terminus of each construct to avoid disrupting myristoylation of the amino terminus. No colonies were obtained after G418 selection when cells were stably transfected with either FLAG-Nrdp1 or FLAG-32. Therefore, we employed transient transfection approaches for protein expression to examine the properties of the Nrdp1 protein. No deleterious effects were observed in any transiently transfected cells after 48 hours of expression.

We have examined the expression and solubility of FLAG-Nrdp1 forms in transiently transfected MCF7 human mammary tumor cells. We observed that detection of FLAG-Nrdp1 by immunoblotting was markedly dependent on the presence of the proteasome inhibitor MG132. Other proteasome inhibitors, but not lysosome or calpain inhibitors, also stabilized FLAG-Nrdp1. The N-terminal region (called FLAG-Zn), including the RING finger, B-box and coiled-coil domains, was also significantly stabilized by MG132. In MCF7 cells, the carboxy terminal portion (FLAG-32) was constitutively expressed and only modestly stabilized by the drug. Similar results were obtained when numerous other mammalian cell lines were transfected with Nrdp1 constructs. These observations indicate that Nrdp1 is an intrinsically unstable protein in mammalian cells, and suggest that its instability is mediated by the amino terminal region of the protein. The full-length protein, but neither of the two halves expressed at similar levels, was more easily extracted from cells under partially denaturing conditions than in an NP-40 lysis buffer, suggesting that some of the intact Nrdp1 protein are associated with insoluble cellular components.

### Example 6

### Specificity of Nrdp1 Interaction with ErbB Receptors

To determine whether Nrdp1 interacts specifically with ErbB3 or might also interact with other members of the ErbB family, we took advantage of the stability of the Nrdp1 protein in Sf9 insect cells. We have observed that in contrast with numerous mammalian cell lines, Nrdp1 protein is stably expressed and does not suppress ErbB3 levels in these cells. We have independently expressed each of the known mammalian ErbB receptors without or with FLAG-Nrdp1 in Sf9 cells, immunoprecipitated receptors and blotted precipitates with antibodies to receptors and anti-FLAG epitope. We observed that Nrdp1 could be coimmunoprecipitated with the neuregulin receptors ErbB3 and ErbB4, but could not be coprecipitated with either ErbB2 or EGF receptor.

To confirm the specificity of the interaction we employed a GST fusion of clone 32 in receptor pull-down experiments. A GST fusion of clone 32 was used instead of full-length Nrdp1 because we observed that GST-Nrdp1 is insoluble in bacteria. We have expressed each of the ErbB family members in Sf9 cells (Carraway, K. L., III, et al., Nature, 387:512-16 (1997)), incubated cleared lysates with immobilized GST or GST-32, and analyzed associated receptors by blotting precipitates with receptor-specific antibodies. We again observed that clone 32 is capable of physically interacting with ErbB3 and ErbB4, but not EGF receptor or ErbB2. Taken together these results teach that Nrdp1 interacts specifically with the neuregulin-binding ErbB3 and ErbB4 receptors relative to the other family members, and that the GST-32 construct accurately recapitulates Nrdp1 binding to receptors.

To assess whether the interaction of Nrdp1 with receptors is dependent on receptor activation state, we examined the association of ErbB3 with GST-32 before and after neuregulin-1β (NRG1β) stimulation of MDA-MB-453 human breast cancer cells. Stimulation of these cells with NRG1_ results in the rapid tyrosine phosphorylation of ErbB2 and ErbB3 as revealed by blotting with anti-phosphotyrosine antibodies (Crovello, C. S., et al., J. Biol. Chem., 273:26954-61)), and a tyrosine-phosphorylated band may be pulled down from lysates of NRG1β-treated MDA-MB-453 cells with GST-32. Reprobing with anti-ErbB3 revealed that similar amounts of this receptor associated with GST-32 whether or not cells were stimulated with NRG1β. These results are consistent with the original yeast two-hybrid screen where the clone 32-encoded polypeptide recognized ErbB3 in its inactive state. Similar results were obtained with ErbB4 expressed in Sf9 cells, where binding of GST-32 to this receptor was independent of NRG1_ stimulation. These results teach that Nrdp1 is capable of interacting with neuregulin receptors independent of receptor activation state and that it could play a role in constitutive receptor activities such as routing or localization. Attempts to co-immunoprecipitate Nrdp1 and neuregulin receptors from co-expressing mammalian cells were unsuccessful because Nrdp1 expression very potently stimulates the loss of neuregulin receptors from cells.

### Example 7

### Co-Localization of ErbB3 and Nrdp1

Despite their inability to be co-immunoprecipitated, confocal immunofluorescence microscopy revealed that ErbB3 and Nrdp1 are very extensively co-localized in transfected COS7 cells, and that Nrdp1 changes the cellular location of ErbB3. COS7 cells were transfected with ErbB3 alone, FLAGNrdp1 alone, or the two proteins together, and then treated with 2 μM MG132 to stabilize protein expression to facilitate visualization. The localization of ErbB3 and Nrdp1 was then examined using indirect immunofluorescence microscopy with anti-ErbB3, anti-Nrdp1 and anti-FLAG antibodies.

ErbB3 on the surface of cells expressing ErbB3 alone exhibited a diffuse, predominantly cell surface distribution, and no significant immunoreactivity was observed in focal planes corresponding to internal structures. Focal planes corresponding to the cell interior revealed a largely punctate perinuclear distribution of FLAG-Nrdp1 when visualized with either antibody. Relatively little Nrdp1 was found at the cell surface in these cells. An untagged version of Nrdp1 exhibited an identical localization pattern in transfected COS7 cells. These observations reveal that Nrdp1 accumulates in intracellular organelles in MG132-treated cells, and the merged images teach that the antiNrdp1 antibody accurately reflects the distribution of the expressed protein.

Cotransfectants exhibited the same perinuclear staining of both proteins as observed with FLAGNrdp1 alone. Identical results were obtained when the experiment was performed in the absence of MG132, and experiments using transfected ErbB2 revealed that Nrdp1 expression had no effect on its localization. These observations indicate that Nrdp1 expression specifically induces a redistribution of ErbB3 from the cell surface to intracellular Nrdp1-containing compartments.

### Example 8

### Nrdp1 Suppresses Cellular Neuregulin Receptor Levels

In the immunofluorescence experiments we noted that the number of ErbB3-expressing cells was reduced by 75-90% when ErbB3 was co-expressed with FLAG-Nrdp1 relative to when expressed on its own. Moreover, it has been demonstrated that RING finger domain-containing proteins serve as E3 ubiquitin ligases that mediate the conjugation of ubiquitin to specific cellular targets (Joazeiro, C. A., et al., Cell, 102:549-552 (2000); Jackson, P. K., et al., Trends Cell Biol., 10:429-39 (2000); Joazeiro, C. A., et al., Science, 286:309-12 (1999); Levkowitz, G., et al., Mol. Cell, 4:1029-40 (1999); and Lorick, K. L., III, et al., Proc. Natl. Acad. Sci. USA, 96:11364-11369)). Two RING finger proteins, c-cb1 and Siah, are known to influence cellular levels of plasma membrane proteins by marking them for degradation through ubiquitination. Hence, we believe one functional consequence of the interaction between Nrdp1 and neuregulin-binding ErbB receptors could be the suppression of receptor levels.

To determine whether Nrdp1 might influence cellular neuregulin receptor levels, we utilized the COS7 cell system to transiently co-express FLAG-Nrdp1 and ErbB receptors. COS7 cells express abundant EGF receptor, but little or no ErbB2, ErbB3 or ErbB4. We transiently transfected COS7 cells with each of these receptors, or co-transfected cells with each of the receptors together with FLAG-Nrdp1. The experiment was carried out in the presence of 2 μM MG132, which stabilizes receptor expression in these cells. Lysates of each transfection were blotted with anti-receptor and anti-FLAG antibodies. We observed that FLAG-Nrdp1 expression had no discernable impact on the steady state levels of endogenous EGF receptor, as expected, or on transfected ErbB2, but markedly reduced levels of transfected ErbB3 and ErbB4. This was not due to an effect of Nrdp1 on receptor transcription because ErbB2 was expressed using the same expression plasmid as ErbB4. Moreover, Nrdp1 had no effect on transfected EGF receptor in MCF7 cells, a cell line that contains no endogenous EGF receptor. These results are consistent with the specific association of Nrdp1 and clone 32 with ErbB3 and ErbB4, and reveal that the functional outcome of Nrdp1/receptor interaction is to suppress levels of neuregulinbinding receptors in cells.

### Example 9

### Potentiation of Neuregulin Signaling by a Putative Dominant-Negative Nrdp1

We observed that the presence of very modest levels (2 μM) of MG132 dramatically stabilized ErbB3 in transiently transfected COS7 cells (FIG. 6A), pointing to the existence of a proteasome-dependent process that regulates receptor steady-state levels. The MG132-mediated stabilization of ErbB3 could be overcome by the expression of FLAG-Nrdp1, suggesting that this protein can complement the existing system for ErbB3 removal. Identical results were obtained using transfected MCF7 human mammary tumor cells and 293 human embryonic kidney cells. Interestingly, FLAG-32 stabilized the presence of ErbB3 both in the absence and presence of MG132, while FLAG-Zn had no effect on receptor steady state levels.

These observations indicate that the polypeptide encoded by clone 32 interferes with the mechanism of ErbB3 removal, perhaps by acting as a dominant-negative inhibitor of the process. The stabilizing effect of clone 32 on ErbB3 levels permitted us to examine the effect of Nrdp1 on neuregulin signaling in a transient transfection assay in MCF7 cells. These cells express modest levels of ErbB2 and ErbB3, and respond in an all-or-nothing manner to treatment with NRG1β.

Cells were transfected with epitope (HA)-tagged Erk1, a downstream effector of neuregulin stimulation in receptor-expressing cells, and co-transfected with either plasmid alone or FLAG-32. Cells were then treated with increasing amounts of NRG1β, and lysates were blotted with anti-phosphoErk1/2 to assess the impact of clone 32 expression on Erk activity. NRG1_ potently stimulated the phosphorylation of both endogenous Erk1/2 and HA-tagged Erk1. The presence of 32 only very modestly enhanced the response of endogenous Erk1/2 to growth factor, but significantly enhanced (2-2.5 fold after quantification) the response of HA-Erk1 to NRG1β. Hence, as expected because of the low transfection efficiency (10-20%) transiently transfected 32 had a marginal effect on the total cellular Erk1/2 population and preferentially acted on the transfected population. Blotting with anti-HA antibodies showed similar levels of the transfected protein in lysates, and blotting with anti-FLAG showed FLAG-32 expression only in transfected cells.

These results reveal that the polypeptide encoded by clone 32 potentiates NRG1β signaling through receptors.

The maintenance of a narrow range of cell surface growth factor receptor tyrosine kinases (RTKs) at specific sites of signal reception ensures sufficient signaling for a proper cellular response, while preventing over-signaling that could lead to disease (Carraway and Sweeney, 2001). In this study we identify and characterize a novel protein that may be involved in this process. We observed that Nrdp1 expression elicits the redistribution of neuregulin receptors from the cell surface to punctate perinuclear intracellular compartments, and induces a loss of receptor expression in cells. These processes are independent of growth factor binding and receptor activation. Moreover, a putative dominant-negative form enhances receptor expression in cells and potentiates NRG1-stimulated Erk1 activity, revealing that Nrdp1 functions to regulate steady-state cell surface receptor levels and neuregulin signaling efficiency.

Nrdp1 is a member of a subfamily of RING finger domain-containing proteins called the tripartite motif family (Borden, K. L., III, et al., Biochem. Cell Biol., 76:351-58 (1998)) or RBCC for RING, B-box, coiled-coil. This subfamily consists of over two-dozen proteins thought to be involved in a variety of developmental and cellular processes, and mutation or rearrangement of some RBCC genes is associated with human disease (Torok, M., et al., Differentiation, 67:63-71 (2001)). RING fingers are zinc-binding domains thought to mediate a variety of protein-protein interactions, and are found in a subclass of E3 ubiquitin ligases (Lorick, K. L., III, et al., Proc. Natl. Acad. Sci. USA, 96:11364-69 (1999)). B-boxes, also called TRAF-type zinc fingers, are zinc-binding domains of unknown function. Coil-coil regions also mediate protein-protein interactions, and often mediate the homotypic dimerization of a protein.

Taken together with its structure, our results are most consistent with a model where Nrdp1 mediates the ubiquitination and trafficking of neuregulin receptors to intracellular degradative compartments. It is tempting to draw parallels between the activity of Nrdp1 toward ErbB3 and ErbB4, and the activity of c-cb1 toward EGF receptor. Upon ligand stimulation, EGF receptor is internalized through clathrin-coated pits and is delivered to intracellular degradative compartments. It has been proposed that EGF stimulation of the EGF receptor results in the autophosphorylation of a specific tyrosine residue, which mediates the recruitment of c-cb1 to the receptor through its tyrosine kinase binding domain. c-cb1 then mediates the ubiquitination of the receptor, which in turn routes the receptor to lysosomes for degradation (Levkowitz, G., et al., Mol. Cell, 4:1029-40 (1999)). In contrast Nrdp1 acts on receptors independent of their activation state. In this regard, it is interesting that the neuregulin target receptors ErbB2, ErbB3 and ErbB4 exhibit impaired ligand-induced internalization, downregulation and degradation (Baulida, J., et al., J. Biol. Chem., 271:5251-57 (1996) and Baulida, J., et al., Exp. Cell Res., 232:167-172 (1999)). The absence of down-regulation, mechanisms that regulate receptor levels could play a major role in defining receptor signaling activity.

Our observations more closely parallel the impact of the RING finger protein Siah on cell surface levels of the membrane protein DCC (deleted in colorectal cancer), a transmembrane receptor for the axon guidance and neuronal migration factor netrin-1. Siah is localized to cytoplasmic particles and mediates the ubiquitination and degradation of DCC in a ligandindependent manner (Hu, G., et al., Genes Dev., 11:2701-14 (1997)). Similar to Nrdp1, Siah is also intrinsically unstable and a putative dominant-negative form stabilizes DCC (Hu, G., et al., Mol. Cell. Biol., 19:724-32 (1999)). Hence, a family of functionally related proteins could mediate the cell surface expression of a variety of proteins.

Future studies will be aimed at more precisely defining the mechanism of receptor degradation, including the roles of receptor and Nrdp1 ubiquitination. It will also be of interest to determine whether Nrdp1 might influence the levels of other proteins, particularly in cardiac, neural and skeletal muscle tissues. Finally, tissue-specific knockout studies should uncover the physiological role of Nrdp1 in regulating the development of neuregulin-dependent tissues.

Although the invention has been described with reference to the presently preferred embodiment, it should be understood that various modifications can be made without departing from the spirit of the invention. Accordingly, the invention is limited only by the following claims.

What is claimed is:

1. An isolated polypeptide comprising an Nrdp1 amino acid sequence consisting of an ErbB3 binding domain of Nrdp1, wherein the Nrdp1 amino acid sequence is not adjacent to an amino acid sequence to which it is naturally adjacent.

2. The isolated polypeptide of claim 1, wherein the ErbB3 binding domain of Nrdp1 is a Nrdp1 fragment lacking a domain selected from the group consisting of the RING finger domain, the B-box domain, and the coiled-coil domain of Nrdp1.

3. The isolated polypeptide of claim 1, wherein the ErbB3 binding domain of Nrdp1 is a Nrdp1 fragment lacking any two domains selected from the group consisting of the RING finger domain, the B-box domain, and the coiled-coil domain of Nrdp1.

4. The isolated polypeptide of claim 1, wherein the ErbB3 binding domain of Nrdp1 is a Nrdp1 fragment lacking the RING finger domain, the B-box domain, and the coiled-coil domain of Nrdp1.

5. The isolated polypeptide of claim 1, wherein the ErbB3 binding domain of Nrdp1 includes at least 183 C-terminal amino acids of Nrdp1.

6. The isolated polypeptide of claim 1, wherein the ErbB3 binding domain of Nrdp1 has an amino acid sequence as shown in SEQ ID NO. 1.

7. The isolated polypeptide of claim 1, wherein the ErbB3 binding domain of Nrdp1 includes at least 138 C-terminal amino acids of Nrdp1.

8. The isolated polypeptide of claim 1, wherein the ErbB3 binding domain of Nrdp1 has an amino acid sequence as shown in SEQ ID NO. 2.

9. An isolated polynucleotide sequence encoding the isolated polypeptide of claim 1.

10. A vector comprising a polynucleotide sequence encoding the isolated polypeptide of claim 1.

11. A cell comprising the isolated polypeptide of claim 1.

12. A cell comprising the isolated polynucleotide sequence encoding the isolated polypeptide of claim 1.

13. A composition comprising the isolated polypeptide of claim 1 and a pharmaceutically acceptable carrier.

14. A composition comprising the isolated polynucleotide of claim 9 and a pharmaceutically acceptable carrier.

15. A composition comprising Nrdp1 and a pharmaceutically acceptable carrier.

16. A composition comprising a polynucleotide encoding Nrdp1 and a pharmaceutically acceptable carrier.

17. A method for decreasing the activity of ErbB3 or ErbB4 in a cell comprising contacting the cell with Nrdp1.

18. The method of claim 17, wherein Nrdp1 does not cause a detectable change of the activity of EGF receptor or ErbB2.

19. The method of claim 17, wherein the amount of ErbB3 is decreased on the surface of the cell.

20. The method of claim 17, wherein the amount of ErbB3 or ErbB4 is decreased in the cell.

21. A method for decreasing the activity of ErbB3 or ErbB4 in a cell comprising introducing a polynucleotide sequence encoding Nrdp1 to the cell.

22. A method for treating the neoplastic growth of a cell comprising contacting the cell with Nrdp1.

23. A method for treating the neoplastic growth of a cell comprising introducing a polynucleotide sequence encoding Nrdp1 to the cell.

24. The method of claim 22, wherein the cell is HER2/neu-positive.

25. A method for treating a neoplastic growth comprising administering to a subject in need of such treatment an effective amount of Nrdp1.

26. The method of claim 25, wherein the neoplastic growth is a solid tumor.

27. The method of claim 26, wherein the solid tumor is HER2/neu-positive.

28. The method of claim 27, wherein the solid tumor is selected from the group consisting of breast, ovary, prostate and lung tumors.

29. A method for treating a neoplastic growth comprising introducing to a subject in need of such treatment a polynucleotide sequence encoding Nrdp1.

30. A method for increasing neuregulin stimulation in a cell comprising contacting the cell with the isolated polypeptide of claim 1.

31. A method for increasing neuregulin stimulation in a cell comprising introducing the isolated polynucleotide of claim 9.

32. A method of screening for an agent capable of regulating an interaction between ErbB3 and Nrdp1 comprising contacting ErbB3 and Nrdp1 in the presence of a test agent, wherein a change in the interaction between ErbB3 and Nrdp1 caused by a test agent is indicative that the agent is capable of regulating the interaction between ErbB3 and Nrdp1.

33. A method of screening for an agent capable of regulating an interaction between Nogo-A and Nrdp1 comprising contacting Nogo-A and Nrdp1 in the presence of a test agent, wherein a change in the interaction between Nogo-A and Nrdp1 caused by a test agent is indicative that the agent is capable of regulating the interaction between Nogo-A and Nrdp1.

34. The method of claim 32, wherein the interaction is binding between ErbB3 and Nrdp1.

35. The method of claim 32, wherein the agent causes increase of the interaction between ErbB3 and Nrdp1.

36. A method for increasing nerve growth comprising administering to a subject in need of such treatment an effective amount of Nrdp1.

37. A method for increasing nerve growth comprising introducing to a subject in need of such treatment a polynucleotide sequence encoding Nrdp1.

38. A method for inhibiting the activity of Nogo-A in a cell comprising contacting the cell with Nrdp1.

39. A method for inhibiting the activity of Nogo-A in a cell comprising introducing a polynucleotide sequence encoding Nrdp1 to the cell.


SEQUENCE LISTING (4 sequences)

SEQ ID NO: 1 - protein, 183 aa, Homo sapiens
IKHLRSVVQQQQTRIAELEKTSAEHKHQLAEQKRDIQLLKAYMRAIRSVNPNLQNLEETIEYNEILEWVNSLQPARVTRWGGMISTPDAVLQAVIKRSLVESGCPASIVNELIENAHERSWPQGLATLETRQMNRRYYENYVAKRIPGKQAVVVMACENQHMGDDMVQEPGLVMIFAHGVEEI

SEQ ID NO: 2 - protein, 138 aa, Homo sapiens
IRSVNPNLQNLEETIEYNEILEWVNSLQPARVTRWGGMISTPDAVLQAVIKRSLVESGCPASIVNELIENAHERSWPQGLATLETRQMNRRYYENYVAKRIPGKQAVVVMACENQHMGDDMVQEPGLVMIFAHGVEEI

SEQ ID NO: 3 - protein, 317 aa, Homo sapiens
MGYDVTRFQGDVDEDLICPICSGVLEEPVQAPHCEHAFCNACITQWFSQQQTCPVDRSVVTVAHLRPVPRIMRNMLSKLQIACDNAVFGCSAVVRLDNLMSHLSDCEHNPKRPVTCEQGCGLEMPKDELPNHNCIKHLRSVVQQQQTRIAELEKTSAEHKHQLAEQKRDIQLLKAYMRAIRSVNPNLQNLEETIEYNEILEWVNSLQPARVTRWGGMISTPDAVLQAVIKRSLVESGCPASIVNELIENAHERSWPQGLATLETRQMNRRYYENYVAKRIPGKQAVVVMACENQHMGDDMVQEPGLVMIFAHGVEEI

SEQ ID NO: 4 - protein, 8 aa, Artificial sequence
  FLAG epitope
DYKDDDDK